(19) United States
(12) Patent Application Publication
(10) Pub. No.: US 2024/0230645 A9
(43) Pub. Date: Jul. 11, 2024

(54) PROTEIN CAPTURE FROM RAW CELL CULTURE USING PROTEIN A AFFIXED IN OPEN TUBULAR AND ANNULAR HELICALLY COILED TUBES

(71) Applicant: Waters Technologies Corporation, Milford, MA (US)

(72) Inventor: Lindsay Morrison, Waltham, MA (US)

(73) Assignee: Waters Technologies Corporation, Milford, MA (US)

(21) Appl. No.: 18/490,562

(22) Filed: Oct. 19, 2023

Related U.S. Application Data
(60) Provisional application No. 63/418,112, filed on Oct. 21, 2022.

(51) Int. Cl. G01N 33/566; G01N 33/543

(52) U.S. Cl. CPC G01N 33/566; G01N 33/5436

(57) ABSTRACT

Provided herein is a tubular protein capture device including a continuous tubular body having an inner surface and an outer surface and methods of using thereof for cell clarification purposes. A protein selected from protein A, protein G, protein L or combination thereof is attached to the inner surface of the tubular protein capture device described herein. The devices and methods provided herein are useful for improving the efficiency and/or capacity of cell clarification to separate proteins. In some embodiments, the continuous tubular body of the present technology has a helical shape. In other embodiments, the continuous tubular body of the present technology includes a plurality of tubes aligned within a hollow enclosure.

### CROSS-REFERENCED TO RELATED APPLICATIONS

This application claims priority to U.S. Provisional Application No. 63/418,112 filed on Oct. 21, 2022, the entirety of which is incorporated herein by reference.

### FIELD OF THE TECHNOLOGY

The present technology generally relates to a cell clarification device and technique to separate proteins from a sample matrix e.g., raw cell culture or lysed cell culture. Particularly, the present technology relates to a tubular protein capture device including a continuous tubular body having an inner surface and an outer surface and methods using thereof. The tubular protein capture device of the present technology includes a protein selected from protein A, protein G, protein L or combination thereof attached to the inner surface of the tubular device.

### BACKGROUND

Biological macromolecules such as proteins constitute an important class of products in the food, biotechnology, pharmaceutical, and cosmetics industries. Typically, proteins are produced by cell culture, using either mammalian or bacterial cell lines engineered to produce the protein of interest by insertion of a recombinant plasmid containing the gene for that protein. Since the cell lines used are living organisms, they must be fed with a complex growth medium, containing sugars, vitamins, amino acids, and growth factors, often supplied from preparations of animal serum. Therefore, separation of the desired proteins from the mixture of compounds fed to the cells and from the byproducts of the cells themselves to a purity sufficient for analytical characterization poses a formidable challenge. In addition, recent advances in efficiency of bioreactors have significantly increased cell density and cellular debris as well as process and product related impurities. That improved upstream efficiency has led to new purification challenges resulting from high product and contaminant concentrations as well as complex components.

Mechanical filtration is one of the most common strategies for cell removal after lysis of the cells to obtain a clarified sample containing the protein of interest. However, fouling of the filter membranes resulting in formation of filtration cakes at the surface of traditional depth filters can limit the number of repeat sample draws that are clarified. Membrane fouling is a process by which the particles, colloidal particles, or solute macromolecules are deposited or adsorbed onto the membrane pores or onto a membrane surface by physical and chemical interactions or mechanical action, which results in smaller or blocked membrane pores. In addition, mechanical filtration incurs the risk of loss of the target protein due to adsorption of the proteins on the filter and increases the risk of cell shear due to increases in backpressure and pore size reduction.

The growing need to develop efficient, rapid and cost-effective protein purification methods is driving research and growth in this area. New strategies for clarification of proteins and methods of pretreatment can improve clarification efficiency. Such approaches lead to better purity of the protein, improving the overall efficiency of downstream purification steps that follow.

Tangential flow filtration is advantageous for bioreactor clarification as the permeate stream can be introduced directly to the subsequent product capture step. However, its use is limited to larger scale production and is difficult to efficiently implement on the analytical scale due to the small volumes needed for sample draws.

### SUMMARY

One of the objectives of the present disclosure is to improve the efficiency and/or the capacity of cell clarification to separate proteins.

The present technology allows selective capture of proteins from raw cell culture as well as lysed cell culture. Capturing proteins from raw cell culture may lead to decrease in the number of steps in protein purification processes, thereby providing increases in efficiency and lowering overall costs.

In one aspect, the present disclosure is directed to a tubular protein capture device, comprising (i) a continuous annular tubular body having an inner surface and an outer surface, wherein the inner surface defines a fluid flow path and includes a protein attached to at least a portion of the inner surface, the protein selected from protein A, protein G, protein L, or combination thereof; and (ii) an inlet and an outlet for flowing a sample matrix, the inlet and outlet being in fluid communication with the fluid flow path, wherein the continuous tubular body has a helical shape that extends continuously from the inlet to the outlet of the continuous annular tubular body

In another aspect, the present technology is directed to a tubular protein capture device, including (i) a continuous tubular body having an inner surface and an outer surface, wherein the inner surface defines a fluid flow path and includes a protein attached to at least a portion of the inner surface, the protein selected from protein A, protein G, protein L, or combination thereof; and (ii) an inlet and an outlet for flowing a sample matrix, the inlet and outlet being in fluid communication with the fluid flow path.

In some embodiments, the continuous tubular body has a helical shape that extends continuously from the inlet to the outlet of the continuous tubular body. In some embodiments, the continuous tubular body has an annular geometry. In some embodiments, the continuous tubular body has an open tubular geometry.

In some embodiments, the protein attached to the inner surface of the tubular protein capture device is selected from protein A, protein G, protein L or combination thereof. In some embodiments, the protein attached to the inner surface of the tubular protein capture device is protein A. In some embodiments, the protein is functional derivative, fragment, or variant of protein A

The above aspects may include one or more of the following features. In some embodiments, the sample matrix comprises at least one type of protein. In some embodiments, the sample matrix comprises a cell culture, a cellular material, a cell extract or combination thereof.

In some embodiments, the sample matrix is a clarified sample. That is, the clarified sample has been subjected to a clarification step before e.g., a centrifugation. In some embodiments, the sample matrix is lysed.

In some embodiments, the sample matrix is in the form of a solution. In some embodiments, the solution is heterogenous. In some embodiments, the solution is homogenous.

In some embodiments, the sample matrix is substantially free of cells. In other embodiments, the sample matrix is a raw cell culture.

In other embodiments, the continuous tubular body comprises a plurality of tubes aligned within a hollow enclosure, wherein the inlet and the outlet for flowing a sample e.g., a solution being in fluid communication with the plurality of tubes.

In some embodiments, the inner surface of the continuous tubular body e.g., at least a portion of the inner surface in fluid communication with a sample is coated with a polymer, and the protein (i.e., capture protein) selected from protein A, protein G, protein L or combination thereof is attached to the polymer. In some embodiments, the protein is selected from functional derivative, fragment, or variant of protein A. In some embodiments, the polymer comprises polyethylene glycol, fluorinated ethylene propylene or ethylene tetrafluoroethylene. In some embodiments, the polymer comprises functional hydroxyl groups. In some embodiments, the protein is attached to the polymer through a covalent bond. In some embodiments, the protein is attached to the polymer through a non-covalent interaction.

In some embodiments, the protein selected from protein A, protein G, protein L or combination thereof is capable of binding to at least one type of protein within the sample matrix.

In some embodiments, the continuous tubular body of the present disclosure is configured to sustain laminar flow at an apparent Reynolds Number between 3 and 60.

In some embodiments, the helical shaped continuous tubular body comprise more than one turns, wherein the distance between consecutive coils (e.g., pitch) is less than 10 mm.

In some embodiments, the outer surface of continuous tubular body disclosed herein is made of a material selected from metal, glass and polymer. In some embodiments, the polymer is selected from a material comprising polyethylene glycol, fluorinated ethylene propylene or ethylene tetrafluoroethylene. In one aspect, the present disclosure is directed to methods for isolating and/or purifying proteins from a sample matrix e.g., lysed cell culture or raw cell culture.

In another aspect, the present technology is directed to a method of capturing at least one type of protein from a sample matrix. The method includes: a) providing the tubular device according to one or more embodiments disclosed herein; b) flowing the sample matrix through the continuous tubular body, wherein the protein selected from protein A, protein G, protein L or combination thereof is capable of binding to at least one type of protein within the sample matrix; c) binding the at least one type of protein within the sample matrix to the protein selected from protein A, protein G, protein L or combination thereof, thereby capturing at least one type of protein from the sample matrix. In some embodiments, the sample matrix comprises a cell culture, a cellular material, a cell extract e.g., a lysed cell culture or combination thereof.

In another aspect, the present technology is directed to a method of extracting at least one type of protein from a sample matrix comprising a cell culture, a cellular material, a cell extract, or combination thereof, including:(i) performing the method of capturing at least one type of protein from the sample matrix according to multiple embodiments of the present disclosure; and (ii) eluting the tubular device with a mobile phase having a pH less than 6.

The above aspects can include one or more of the following features. In some embodiments, the sample matrix comprises less than 100 μg of a total protein.

In some embodiments, the sample matrix flows through the continuous tubular body at an apparent Reynolds Number between 3 and 60.

The devices and methods of the present technology provide numerous advantages. For example, the devices and methods provide improved interaction between the sample matrix and clarification media/material. In particular, a number of embodiments take advantage of the geometry of the flow paths to improve capture of specific proteins. In some embodiments, the present technology allows for tailoring of the Dean number of the fluid flow to improve radial mixing of the sample matrix with the protein coated on the inner surface of the tubular device. The Dean number may be tailored by changing flow rate.

The design of the devices of the present technology e.g., helical design leverages Dean vortices to improve radial mixing. In some embodiments, improved radial mixing increases protein capture efficiency. The design of the devices of the present technology e.g., annular design further provides higher surface area aspect ratios compared to the conventional devices e.g., devices using dead-end filtration technique or devices having open tubular design, resulting in higher protein capture efficiency.

The devices and methods provided herein using open tubular and annular geometries aids minimizing shear forces on cell membranes, leading to decrease in cell rupture.

### BRIEF DESCRIPTION OF DRAWINGS

The technology will be more fully understood from the following detailed description taken in conjunction with the accompanying drawings, in which:

FIG. 1A and FIG. 1B show cross-sectional view of cross-sections of annular (FIG. 1A) and open tubular (FIG. 1B) geometries.

FIG. 1C shows a cross-section of open tubular body of the present technology including a capture protein attached on the inner surface of the open tubular body.

FIG. 1D, FIG. 1E, and FIG. 1F show a cross-section of annular tubular body of the present technology including a capture protein (110) attached on at least a portion of the inner surface of the annular tubular body. Collectively, FIG. 1D-FIG. 1F present different embodiments of the present technology where the capture protein (110) is attached to different portions of the inner surface within the annulus.

FIG. 2 shows change in wall shear rate (s⁻¹) with respect to the changes in volumetric flow rates (μL/min) in a device having open tubular geometry with a 145 μm diameter (diamond), open tubular geometry with a 203 μm diameter (square), annulus with a 51 μm diameter (cross), and open tubular with a 254 μm diameter (triangle).

FIG. 3 shows an example of a substantially coiled continuous tubular body according to multiple embodiments of the present disclosure.

FIG. 4A, FIG. 4B, FIG. 4C, FIG. 4D, FIG. 4E, FIG. 4F, FIG. 4G, and FIG. 4H show various multi-lumen tube configurations according to multiple embodiments of the present disclosure.

FIG. 5A and FIG. 5B shows the effect of flow rate 200 μL/min (FIG. 5A) and 100 μL/min (FIG. 5B) on Dean numbers as well as the effect of coil diameter (10 mm and 5 mm) and internal tube diameter (150 μm, 200 μm, 250 μm) on Dean's number.

FIG. 6 depicts the effect of loading flow rate (1-9 μL/sec) on the binding capacity of an open tubular capture device of the present disclosure.

FIG. 7 depicts the effect of loading flow rate (1-50 μL/sec) on the binding capacity of an annular cross-section protein capture device of the present disclosure.

### DETAILED DESCRIPTION

Following below are more detailed descriptions of various concepts related to, and embodiments of, devices and methods for separating proteins from a sample matrix using, inter alia, a continuous tubular body including a capture protein. The capture protein can selectively bind to or interact with one or more proteins present in a sample matrix. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

The present disclosure provides novel and improved processes for purifying one or more protein(s) of interest from a sample matrix including one or more protein(s) of interest. In certain instances, the devices and methods disclosed decreases the number of steps that may be used in a purification process of proteins, thereby reducing the overall operational costs, and saving time. As used herein, the terms “purifying,” “separating,” or “isolating,” as used interchangeably herein, refer to increasing the degree of purity of a target molecule e.g., a protein from a composition or sample matrix e.g., a solution comprising the target molecule and one or more impurities. Typically, the degree of purity of the target molecule is increased by removing (completely or partially) at least one impurity from the composition.

The methods and devices provided herein are useful for cell clarification. The term “clarification” as used herein, refers to a process for reducing turbidity, as measured in nephelometric turbidity units (NTU), of a protein-containing matrix (e.g., a solution), by removing suspended particles. Clarification can be achieved by a variety of means, including batch and continuous centrifugation, depth filtration, normal and tangential flow filtration, and precipitation, including flocculation with small molecule and polymeric species, or any combinations of methods thereof. The methods disclosed may be performed before or after any clarification technique that is used in the art for cell clarification.

Procedures for purification of proteins from cell debris initially depend on the site of expression of the protein. Some proteins are secreted directly from the cell into the surrounding growth media; others are produced intracellularly and retained within the cell. For the latter proteins, the first step of a purification process involves lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by centrifugation or by filtration. The same problem arises, although on a smaller scale, with directly secreted proteins due to the natural death of cells and release of intracellular host cell proteins in the course of the protein production run. Consequently, typical purification processes that are presently used include the following steps: (i) cell lysis to recover an intracellular protein or recovery of a protein from the media in case of a secreted protein; (ii) removal of cellular debris using e.g., differential centrifugation or filtration to obtain a clarified sample containing the one or more protein(s) of interest and (iii) use of a variety of chromatography media in a multi-step process to separate the protein(s) of interest from other proteins and/or the various other impurities in the sample matrix. The methods and devices of the present disclosure may be performed/used during step (iii). Since the methods and devices disclosed herein allows clarification of raw cell cultures, in some instances, step (i) and (ii) may not be performed. Therefore, the present technology may lead to decrease in number of steps that may be used in a purification process of proteins, thereby increasing protein recovery from a sample matrix.

In various methods using the tubular protein capture device provided herein, the loss in yield of the protein of interest is less than about 20%, less than about 10%, less than about 5% of the total protein amount. In other words, methods provided herein using the protein capture device of the present technology result in 80% or greater, 90% or greater, 95% or greater yield (e.g., 97%, 98%, 99%) of protein of interest, where 100% is the total protein amount.

In some embodiments, the one or more protein(s) of interest is an antibody or an Fc region containing protein. In some embodiments, the antibody is a monoclonal antibody. In other embodiments, the antibody is a polyclonal antibody. In some embodiments, a sample matrix comprising one or more protein(s) of interest is a clarified cell culture feed. In some examples, the clarified cell culture feed is obtained via depth filtration and/or centrifugation. In other examples, the clarified cell culture is obtained via precipitation with a salt, an acid, a polymer, or a stimulus responsive polymer. In some embodiments, a sample matrix comprising one or more protein (s) of interest is a raw cell culture feed. The sample matrix may include a cell culture, a cellular material, a cell extract or combination thereof.

In one aspect, the present technology is directed to a tubular protein capture device, including a continuous tubular body having an inner surface and an outer surface. The inner surface defines a fluid flow path where a sample matrix can flow through along the path. A capture protein selected from protein A, protein G, protein L or combination thereof can bind to (e.g., reversibly) or interact with one or more proteins present in a sample matrix. The tubular protein capture device provided herein also includes an inlet and an outlet for flowing the sample matrix, the inlet and outlet being in fluid communication with the fluid flow path.

As used herein, and unless stated otherwise, the term “sample” refers to any composition or mixture that contains one or more protein(s) of interest. Samples may be derived from biological or other sources. Biological sources include eukaryotic and prokaryotic sources, such as plant and animal cells, tissues and organs. The sample may also include diluents, buffers, detergents, and contaminating species, debris and the like that are found mixed with the one or more protein(s) of interest. The sample may be “partially purified” (i.e., having been subjected to one or more purification steps, such as filtration steps) or may be obtained directly from a host cell or organism producing the one or more protein(s) of interest (e.g., the sample may comprise harvested cell culture fluid).

As used herein, the term “continuous body” refers to a physical structure that has an uninterrupted extension in space.

As used herein, the term “includes” means includes but is not limited to, and the term “including” means including but not limited to.

The material selected and the size and shape of the continuous body of annular tube described herein is selected to achieve protein capture from flowing sample matrix including, but not limited, to raw cell culture, lysed cell culture, cell extract, clarified cell culture. As an example, the continuous body of annular tube can be configured in any shape and/or size that is capable of capturing or separating an amount of a protein from a sample matrix. The total amount of the protein within the sample matrix may be less than 900 μg, 800 μg, 700 μg, 600 μg, 500 μg, 400 μg, 300 μg, 200 μg, 100 μg, 50 μg, 25 μg or 10 μg.

In one example, the continuous tubular body of protein capture device may be substantially coiled in shape (e.g., formed in a configuration of concentric rings). In other examples, the continuous tubular body may be substantially non-uniform in shape, i.e., include coiled and/or straight portions that extend in a plurality of differing directions being defined by radial curvatures that differ from each other from the inlet to the outlet of the continuous body.

In another example, the continuous tubular body may be substantially straight in shape including a plurality of tubes aligned within a hollow enclosure. In any example according to the principles herein, the continuous tubular body can be formed in an entangled conformation. As used herein, the term “entangled” refers to a body of tubing having at least a portion that includes a plurality of coils, folds, and/or loops. In an example, the plurality of coils, folds, and/or loops can be overlapping. Such an entangled body of tubing can be obtained, at least in part, by positioning and fabricating the tubing, in a plurality of directions. An example entangled tubing herein can exhibit greatly increased surface area of the resulting body of tubing which may lead to increase in capture efficiency.

As a non-limiting example, the entangled body of tubing may be comprised of continuous small radius bends. In one embodiment, the entangled body of tubing may have an overall compact structure.

Curved and spiral microchannels have a wide range of applications in microfluidic devices such as isolation of circulating tumor cells, cell enrichment, bacteria separation, fluid mixing, microfiltration, and inertial focusing and separation of particles. Fluids flowing in the axial direction of a curved channel experience a pressure gradient along the radial direction. Without wishing to be bound by theory, it is believed that curved fluid passageways lead to continuous circulation of the fluid in the radial direction and subsequent formation of two or more counter-rotating vortices in the channel. This lateral recirculating flow is called a secondary (or Dean) flow. Formation of Dean flow in curved and spiral channels is inevitable, yet their presence may be dominant and useful in some applications while undesirable in others.

The tubular protein capture device of the present disclosure requires maximizing Dean flow while promoting radial mixing to increase capture efficacy of capture proteins that are attached to the inner surface of the tubular protein capture device.

The tubular protein capture device of the present disclosure leverages Dean vortices stimulated by coiled helical geometries to improve radial mixing. Attachment of capture proteins on the inner surface of a coiled tube permits free flow of cellular material to waste while simultaneously optimizing radial mixing and protein capture along the inner surface of the coiled tube.

### CAPTURE PROTEIN(S)

The continuous tubular body of the present disclosure includes a protein e.g., a capture protein that is capable of binding (e.g., reversibly) or interacting with one or more proteins of interest present in a sample matrix. The capture proteins may selectively bind to the protein of interest among other proteins and/or other impurities. The capture proteins of the present disclosure are attached to an inner surface of the tubular protein capture device.

In some instances, the capture proteins may be attached to the inner surface of the tubular body through a polymer which coats the inner surface of the continuous tubular body. That is, in some instances, the inner surface of the tubular body is coated with a polymer and the capture protein is attached to the polymer through a covalent bond, a non-covalent bond or combination thereof.

In some embodiments, the capture protein is attached via a single point attachment to a polymer that coats the inner surface of the continuous tubular body. Single point attachment generally means that the protein moiety is attached via a single covalent bond to a polymer. Such single-point attachment may also occur by use of suitably reactive residues which are placed at an exposed amino acid position, namely in a loop, close to the N- or C-terminus or elsewhere on the outer circumference of the protein fold. Suitable reactive groups are e.g., sulfhydryl or amino functions.

The polymer that coats onto or is affixed onto the inner surface of the continuous tubular body may be any polymer that have free functional groups e.g., —OH, —NH₂, —COOH that can interact or react with a capture protein. In some embodiments, the polymer comprises polyethylene glycol

In some embodiments, the capture protein is selected from protein A, protein G, protein L, a functional variant thereof, or combination thereof. In some embodiments, the protein attached to the inner surface of the tubular protein capture device is protein A. In some embodiments, the protein is functional derivative, fragment, or variant of protein A.

Protein A is a bacterial cell wall protein from Staphylococcus aureas that binds to mammalian IgGs primarily through their Fc regions. In its native state, Protein A has five IgG binding domains as well as other domains of unknown function.

As used herein, the term “immunoglobulin,” “Ig” or “IgG” or “antibody” (used interchangeably herein) refers to a protein having a basic four-polypeptide chain structure consisting of two heavy and two light chains, said chains being stabilized, for example, by interchain disulfide bonds, which can specifically bind antigen. The term “Fc region” and “Fc region containing protein” means that the protein contains heavy and/or light chain constant regions or domains (CH and CL regions as defined previously) of an immunoglobulin. Proteins containing an “Fc region” can possess the effector functions of an immunoglobulin constant domain. An “Fc region” such as CH2/CH3 regions, can bind selectively to affinity ligands such as Protein A or functional variants thereof. In some embodiments, an Fc region containing protein specifically binds Protein A or a functional derivative, variant or fragment thereof. In other embodiments, an Fc region containing protein specifically binds Protein G or Protein L, or functional-derivatives, variants or fragments thereof

Protein A may be recovered from a native source thereof, Protein A may be produced synthetically (e.g., by peptide synthesis or by recombinant techniques). A functional derivative, fragment or variant of Protein A according to the present disclosure may be characterized by a binding constant of at least K=10⁻⁸ M, and preferably K=10⁻⁹ M, for the Fc region of mouse IgG2a or human IgG1. An interaction compliant with such value for the binding constant is termed “high affinity binding” in the present context. Preferably, such functional derivative or variant of Protein A comprises at least, part of a functional IgG binding domain of wild-type Protein A, selected from the natural domains E, D, A, B, C or engineered mutants thereof which have retained IgG binding functionality.

“Binding” refers to an affinity between two molecules, for example, a capture protein and a target protein i.e., a protein of interest. When binding occurs between the protein of interest and the capture protein of the present disclosure, the protein of interest is reversibly immobilized on the capture ligand by virtue of ligand—protein interactions. Non-limiting examples of ligand—protein interactions include antibody-antigen binding, enzyme-substrate binding, enzyme-cofactor binding, metal ion chelation, DNA binding protein-DNA binding, regulatory protein-protein interactions, and the like. The binding may be due to the combined effects of spatial complementarity of e.g., protein of interest and capture protein at a binding site coupled with electrostatic forces, hydrogen bonding, hydrophobic forces, and/or van der Waals forces at the binding site. Generally, the greater the spatial complementarity and the stronger the other forces at the binding site, the greater will be the binding specificity of a protein for its respective ligand.

In some instances, the binding constant between the capture protein and the one or more protein of interest is between K=10⁻⁴ M and K=10⁻⁹ M.

### A TUBULAR PROTEIN CAPTURE DEVICE

In one aspect, provided herein is a tubular protein capture device, including a continuous tubular body having an inner surface and an outer surface, wherein the inner surface defines a fluid flow path and includes a capture protein attached to the inner surface; and (ii) an inlet and an outlet for flowing a sample matrix e.g., a solution, the inlet and outlet being in fluid communication with the fluid flow path.

In some embodiments, the outer and/or the inner surface of continuous tubular body is made of a material selected from metal, glass, polymer and combination thereof. In some embodiments, the metals are selected from titanium, nickel, copper, brass and stainless steel. In some embodiments, the polymer is selected from a material comprising polyethylene glycol, fluorinated ethylene propylene, ethylene tetrafluoroethylene, or combination thereof.

In some embodiments, the outer and/or the inner surface of continuous tubular body is made of polyether ether ketone.

In some embodiments, the outer and/or the inner surface of continuous tubular body is made of fluorinated compounds selected from ethylene tetrafluoroethylene (ETFE) and fluorinated ethylene propylene (FEP).

In some embodiments, the outer and/or the inner surface of continuous tubular body is made of a polymer made by using a thermoforming processing technique.

The devices of the present technology allow optimizing radial mixing by changing geometrical parameters of the devices disclosed herein. For example, promoting radial mixing (e.g., laminar flow) increases the capturing efficiency of capture proteins since the chance of a protein of interest to bind to or interact with a capture protein increases while the sample matrix flows with a laminar flow with a tailored Dean number.

The open tubular design of the tubular protein capture device according to one or more embodiments of the present disclosure aids minimizing shear forces on cells within a sample compared to the conventional filters e.g., dead-end filters.

In other embodiments, the annular design of the tubular protein capture device provides high surface area aspect ratios which is beneficial to increase capturing efficiency. High surface area aspect ratios would be expected to yield higher capture efficiencies due to increased area per cross section over which the affinity capture protein e.g., protein A, protein G, protein L or combination thereof can be attached.

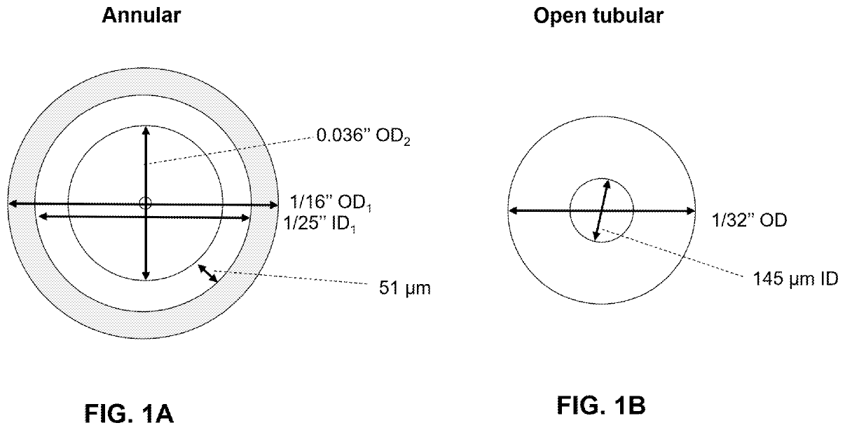

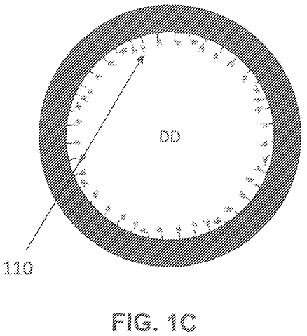

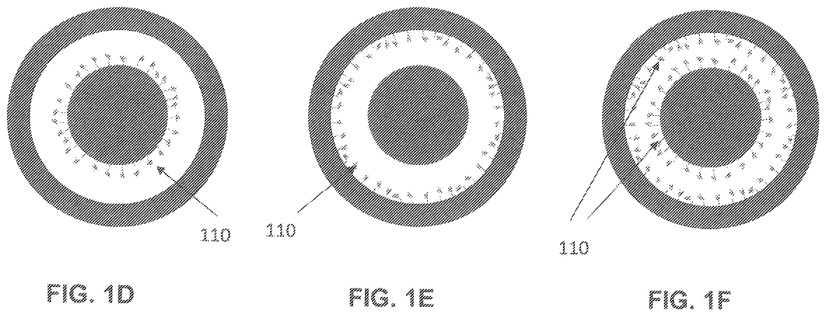

To this end, annular geometry in which the sample matrix is flowed through a tubular design comprised of a cross-sectional ring is expected to yield improved results. Such a design would also benefit from a coiled helical configuration, as Dean vortices are generated across the annulus. FIG. 1A and FIG. 1B show cross-sections of annular (FIG. 1A) and open tubular (FIG. 1B) geometries. The surface aspect ratio of annular design with 51 μm annulus corresponding to FIG. 1A (cross section area of annular capillary: 0.155 mm2; surface area of annular capillary: 6063 μm/L) is 0.0392, whereas the surface aspect ratio of open tubular design with 145 μm internal diameter corresponding to FIG. 1B (cross section area of annular capillary: 0.0177 mm2; surface area of annular capillary: 471 μm/L) is 0.0267. That is, the continuous annular tubular body of the present disclosure provides larger surface area to attach more capture proteins onto the inner surface of the tubular body compared to the open tubular design. Cross-sectional view of continuous tubular body of the present technology is provided in FIG. 1C-1F to show how a capture protein (110), e.g., protein A, protein G, protein L or combination thereof, is attached to the inner surface of the tubular body.

FIG. 1C shows how a capture protein (110) is attached to the inner surface of the open tubular body.

FIG. 1D-1F presents different variations of the present technology where the capture protein is attached to different portions of the inner surface within the annulus geometry of a tubular body. Utilization of a wall-centered approach in which one or more protein(s) of interest is directly captured from a sample matrix eliminates the need for mechanical filtration and associated risks of protein absorption and wall shearing of cells.

As used herein, “annular” or “annulus” refers to the space between two substantially concentric objects (or between two substantially concentric regions such as between casing and tubing, where fluid can flow. Annular shapes suitable for use with the present invention include a ring, an oval, an ellipse, a toroid, and the like.

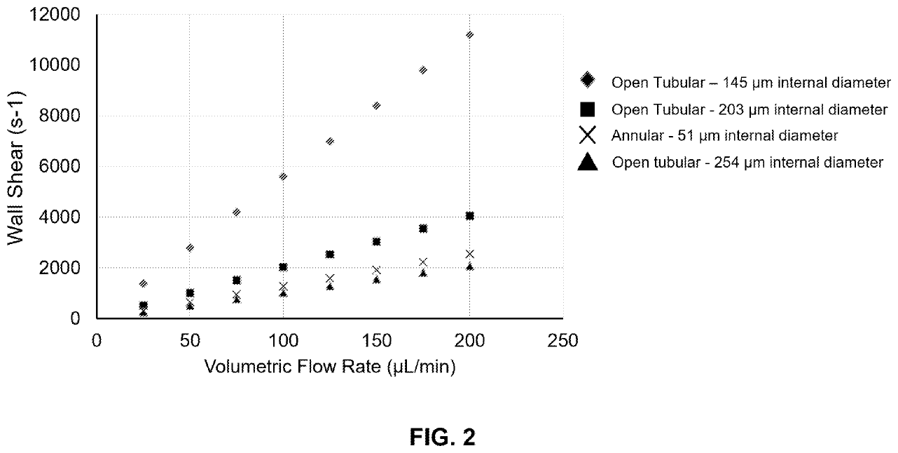

Depending on the internal diameter, both annular and open tubular geometries presented herein help reducing wall shear rates compared to conventional filtration techniques, which results reduced cell rupturing and a subsequent reduction in host cell protein contaminants (FIG. 2). In some embodiments, the continuous tubular body has a helical shape that extends continuously from the inlet to the outlet of the continuous tubular body.

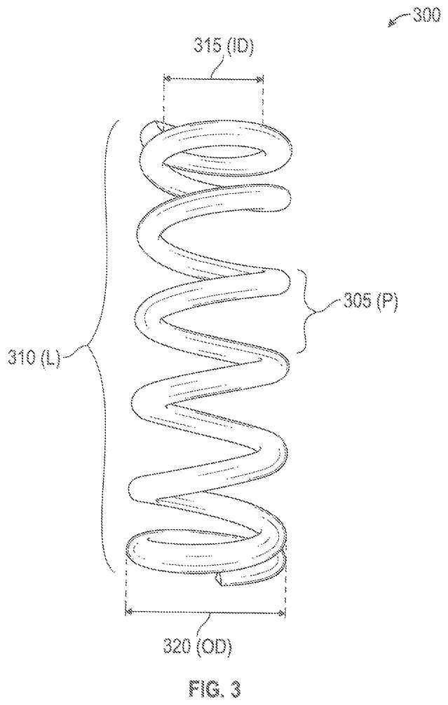

FIG. 3 shows an example of a substantially coiled continuous tubular body 300. As shown in FIG. 3, the diameter of the outer dimension (320 or OD) is greater than the diameter of the inner dimension (315 or ID). With reference to FIG. 3, the outer perimeter wall is located, for example, at OD or 320, whereas the interior perimeter wall is located at ID or 310

As shown in FIG. 3, the continuous tubular body can be characterized by one or more of: the length or distance, L or 310, the distance between consecutive coils (pitch), P or 305.

In various non-limiting example implementations, the continuous tubular body can be formed with a length (L) varying between about 40 mm and about 5mm. Particularly, the length (L) can vary between, about 20 mm and about 1 mm, about 20 mm and about 5 mm. In general, the length can be any length to accommodate system preferences.

In various non-limiting example implementations, the continuous tubular body can be formed with a mean coil diameter varying between about 1 mm and about 20 mm. Particularly, the mean coil diameter can vary between about 5 mm and about 15 mm. More particularly, the mean coil diameter can vary between about 5 mm and about 10 mm. In general, the coil diameter of the tubing is selected in accordance with the flow and resistance needs of a particular system. As a result, a wide variety of inner diameters are available for use.

In various non-limiting example implementations, the continuous tubular body can be formed with a pitch (P) varying between about 1 mm and about 10 mm upon system preference.

In some example implementations, the continuous tubular body illustrated in FIG. 3 can be formed with dimensional parameters (e.g., OD, ID, P) that remain substantially constant over the length, L. In other embodiments, one or more of these dimensions can be varied over the length, L. For example, the pitch P can be varied over the continuous tubular body length L, such that the pitch P exhibits a constant decrease or increase over L, or intermittent decrease or increase over L, or both. As another example, the diameter of the inner dimension ID can be varied over the continuous tubular body length L, such that the variation in ID can be a constant decrease or increase over L, or intermittent decrease or increase over L, or both. As another example, the diameter of the outer dimension OD can be varied over the continuous tubular body length L such that the variation in OD can be a constant decrease or increase over L, intermittent decrease or increase over L, or both. As another example, continuous body of gas-permeable tubing can be formed with both ID and OD increasing over the length of L, i.e., the coil can change in size over L. The percent increase for any one of these dimensions (ID, OD and P) over L can be up to 5%, 10%, 15%, 20%, 30%, 40%, 50%, 60%, 70%, 80%, 90%, or 100%. In one embodiment, the continuous body of gas-permeable tubing can be configured with a tightly coiled flow path such that over L, all three of the dimensional parameters (ID, OD, and P) increase over the length L.

In some embodiments, the continuous tubular body is configured to sustain laminar flow at an apparent Reynolds Number between 2 and 60, between 3 and 50, between 5 and 40, between 5 and 30, between 5 and 20, between 3 and 10, between 3 and 7, between 3 and 60, or between 3 and 60.

In some embodiments, the moving fluid comprises a flow rate of between 100 μL/min and 1250 μL/min. The flow rate of the sample matrix disclosed herein may be adjusted to obtain optimum Dean numbers to promote capturing efficiency.

In some embodiments, the continuous tubular body is configured to sustain laminar flow at a Dean Number between 1.5 and 6, between 1.5 and 5, or between 2 and 4.

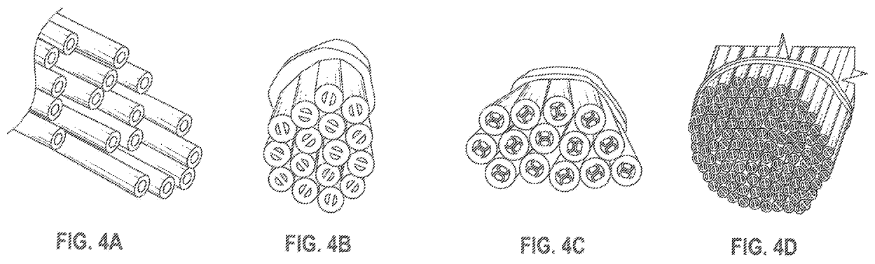

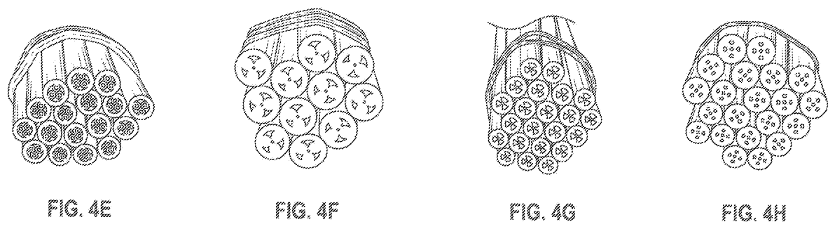

Provided herein is a tubular protein capture device according to multiple embodiments disclosed herein, wherein the continuous tubular body comprises a plurality of tubes aligned within a hollow enclosure, wherein the inlet and the outlet for flowing a solution being in fluid communication with the plurality of tubes (FIG. 4A-H). Inner diameter of each tube within the hollow enclosure can be designed in various geometries e.g., star internal fiber, screw, slot channel (FIG. 4A-H).

In some embodiments, the continuous tubular body may include between about 3 to about 500 tubes within the hollow enclosure of the continuous tubular body. The number of tubes included in the hollow enclosure may vary depending on the inner and/or outer diameter of the tubes within the hollow enclosure of the continuous tubular body as well the diameter of the hollow enclosure.

In some embodiments, each tube is a single-channel tube. That is, there is only one channel in each tube for sample to flow through (FIG. 4A). In other embodiments, each tube within the hollow enclosure of the continuous tubular body has multiple interior lumens. The lumens can be formed using various cross-sectional configurations. For example, FIG. 4A-H illustrates various cross-sectional configurations which result in the incorporation of a single channel (FIG. 4A) (i.e., a single lumen) or a plurality of lumens (e.g., 1 Web providing two distinct lumens (FIG. 4B), 2T, which provides 4 lumens (FIG. 4C), 2W, which provides at least 110 lumens (FIG. 4D), 3T, which provides 17 lumens (FIG. 4E), 3W providing 11 lumens (FIG. 4F), 3Y providing 22 lumens (FIG. G), 4+providing 21 lumens (FIG. 4H)). As used herein, a “lumen” means a channel within a tube.

A multi-lumen tube could provide side-by-side flow of a sample. A multi lumen-tube provides high surface area aspect ratios which is beneficial to increase capturing efficiency.

Lumens within a continuous tube may have different sizes and shapes based upon system preferences (e.g., 1 Web, 2T, 2W, 3T, 3W, 3Y, 4+ as shown in FIG. 4A-H). Multi-lumen tubing provides multiple channels in a single-tube structure.

In various non-limiting example implementations, the tube diameters vary between about 0.1 mm and about 20 mm. Specifically, the tube diameters vary between about 0.15 mm and about 10 mm.

### THE METHOD FOR CLARIFICATION

In one aspect, provided herein is a method of capturing at least one type of protein from a sample matrix using the tubular protein capture device according to multiple embodiments of the present disclosure.

A chromatographic “capture” step, as used herein, includes binding protein of interest to a capture protein. The primary function of this step is to bind the one or more protein of interest from a sample matrix, while allowing the impurities e.g., contaminating proteins, lipids, carbohydrates and the like to flow through. The protein of interest is then eluted with a buffer for further downstream processing.

As used herein, the term “impurity” or “contaminant” as used herein, refers to any foreign or objectionable molecule, including a biological macromolecule such as DNA, RNA, one or more host cell proteins, endotoxins, lipids and one or more additives which may be present in a sample containing the target molecule e.g., a protein that is being separated from one or more of the foreign or objectionable molecules using a process of the present invention. Additionally, such impurity may include any reagent which is used in a step which may occur prior to the methods disclosed herein.

The method provided herein for capturing at least one type of protein from a sample matrix includes (i) providing the tubular device according to multiple embodiments disclosed herein; (ii) flowing the sample matrix through the continuous annular tubular body, wherein the capture protein is capable of binding to at least one type of protein within the sample matrix; (iii) binding the at least one type of protein within the sample matrix to the capture protein, thereby capturing at least one type of protein from the sample matrix.

Further provided herein is a method of extracting at least one type of protein from a sample matrix. After performing the method of capturing at least one type of protein from a sample matrix according to multiple embodiments of the present technology, the tubular device is eluted with a mobile phase having a pH less than 6.9, 6.8, 6.7, 6.6, 6.5, 6.4, 6.3, 6.2, 6.1, 6.0, 5.0, or 4.5. To “elute” a molecule (e.g., a polypeptide of interest or an impurity) from chromatography resin is meant to remove the molecule therefrom by altering the solution conditions such that buffer competes with the molecule of interest for binding to the chromatography resin. A non-limiting example is to elute a molecule from an ion exchange resin by altering the ionic strength of the buffer surrounding the ion exchange material such that the buffer competes with the molecule for the charged sites on the ion exchange material.

In some embodiments, the sample matrix comprises less than 100 μg of a total protein.

In some embodiments, the sample matrix flows through the continuous tubular body at an apparent Reynolds Number between 3 and 60.

In some embodiments, the sample matrix is flowed onto the open tubular body at a loading flow rate of between 3-7 uL/sec. In some embodiments, the loading flow rate is 3, 4, 5, 6, or 7 uL/sec.

In some embodiments, the sample matrix is flowed onto the annular cross-section tubular body at a loading flow rate of between 20-50 uL/sec. In some embodiments, the loading flow rate is 20-25, 25-30, 30-35, 35-40, 40-45, or 45-50 uL/sec.

In some embodiments, the sample matrix flows through the continuous tubular body at Dean number between 2.5 to 6.0, 3.0 to 5.0 or 3.5-4.5.

Dean numbers can be optimized by changing flow rate of the matrix solution.

### EXAMPLES

### Example 1: The Effect of Flow Rate, Coil Diameter and Internal Tube Diameter on Dean's Number

Flow rate, coil diameter and internal tube diameter are derived mathematically and are drawn from the table listed below (Table 1). The values for the Deans numbers were obtained from the following formula:

$$\\ De = Re \times \sqrt{d / {D ( 1 + \frac{ρ}{π D} )}^{{}^{} 2}}$$

Where Re is the Reynolds number, d is the tube diameter, D is the coil diameter, and p is pitch in mm.

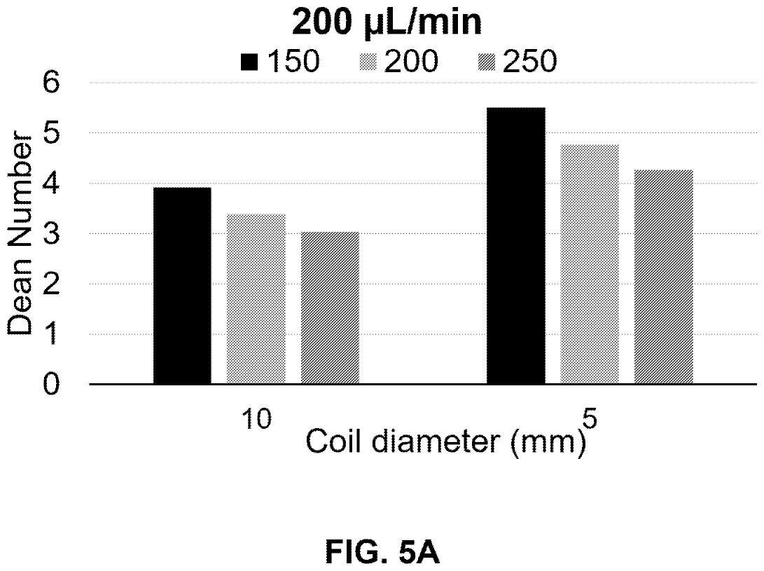

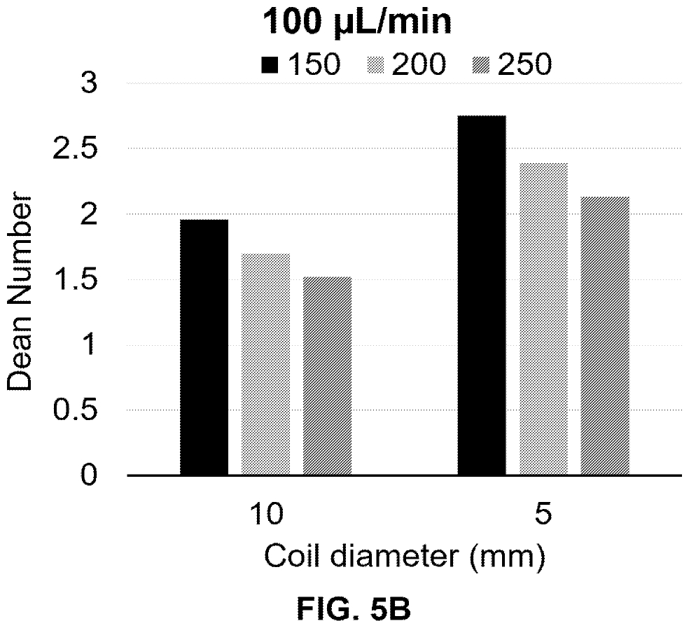

FIG. 5A and FIG. 5B are graphical representations of these calculated values. 10 and 5 presented in FIG. 5A and FIG. 5B refer to the coil diameter in mm; 150, 200, and 250 each correspond to the tube inner diameters in μm; and 200 μL/min and 100 μL/min refer to flow rate. FIG. 5A and FIG. 5B demonstrate that smaller tube and coil diameters generate higher Dean numbers, which is predicted to yield higher recoveries due to there being more contact between the protein and the modified surfaces. In addition, FIG. 5A and FIG. 5B demonstrate that the Dean number increases with volumetric flow rate. This information is useful for considerations around cell shear associated with flow rate.

TABLE 1
Variation of Dean's numbers based
on tube ID, coil D, pitch and flow rate

| Tube | Coil D | Pitch | Flow Rate | Dean |
|---|---|---|---|---|
| ID (um) | (mm) | (mm) | (μl/min) | Numbers |
| 150 | 10 | 1 | 200 | 3.92 |
| 150 | 10 | 2 | 200 | 3.92 |
| 150 | 10 | 4 | 200 | 3.895 |
| 150 | 10 | 8 | 200 | 3.81 |
| 150 | 10 | 1 | 100 | 1.96 |
| 150 | 10 | 2 | 100 | 1.96 |
| 150 | 10 | 4 | 100 | 1.95 |
| 150 | 10 | 8 | 100 | 1.903 |
| 150 | 5 | 1 | 200 | 5.54 |
| 150 | 5 | 2 | 200 | 5.51 |
| 150 | 5 | 4 | 200 | 5.382 |
| 150 | 5 | 8 | 200 | 4.95 |
| 150 | 5 | 1 | 100 | 2.77 |
| 150 | 5 | 2 | 100 | 2.75 |
| 150 | 5 | 4 | 100 | 2.69 |
| 150 | 5 | 8 | 100 | 2.47 |
| 200 | 10 | 1 | 200 | 3.4 |
| 200 | 10 | 2 | 200 | 3.39 |
| 200 | 10 | 4 | 200 | 3.37 |
| 200 | 10 | 8 | 200 | 3.3 |
| 200 | 10 | 1 | 100 | 1.7 |
| 200 | 10 | 2 | 100 | 1.7 |
| 200 | 10 | 4 | 100 | 1.69 |
| 200 | 10 | 8 | 100 | 1.65 |
| 200 | 5 | 1 | 200 | 4.8 |
| 200 | 5 | 2 | 200 | 4.77 |
| 200 | 5 | 4 | 200 | 4.66 |
| 200 | 5 | 8 | 200 | 4.29 |
| 200 | 5 | 1 | 100 | 2.4 |
| 200 | 5 | 2 | 100 | 2.39 |
| 200 | 5 | 4 | 100 | 2.33 |
| 200 | 5 | 8 | 100 | 2.14 |
| 250 | 10 | 1 | 200 | 3.04 |
| 250 | 10 | 2 | 200 | 3.04 |
| 250 | 10 | 4 | 200 | 3.02 |
| 250 | 10 | 8 | 200 | 2.95 |
| 250 | 10 | 1 | 100 | 1.52 |
| 250 | 10 | 2 | 100 | 1.52 |
| 250 | 10 | 4 | 100 | 1.51 |
| 250 | 10 | 8 | 100 | 1.47 |
| 250 | 5 | 1 | 200 | 4.29 |
| 250 | 5 | 2 | 200 | 4.27 |
| 250 | 5 | 4 | 200 | 4.17 |
| 250 | 5 | 8 | 200 | 3.83 |
| 250 | 5 | 1 | 100 | 2.15 |
| 250 | 5 | 2 | 100 | 2.13 |
| 250 | 5 | 4 | 100 | 2.08 |
| 250 | 5 | 8 | 100 | 1.92 |

### Example 2: Open Tubular and Annular Cross-Section Loading Flow Rates

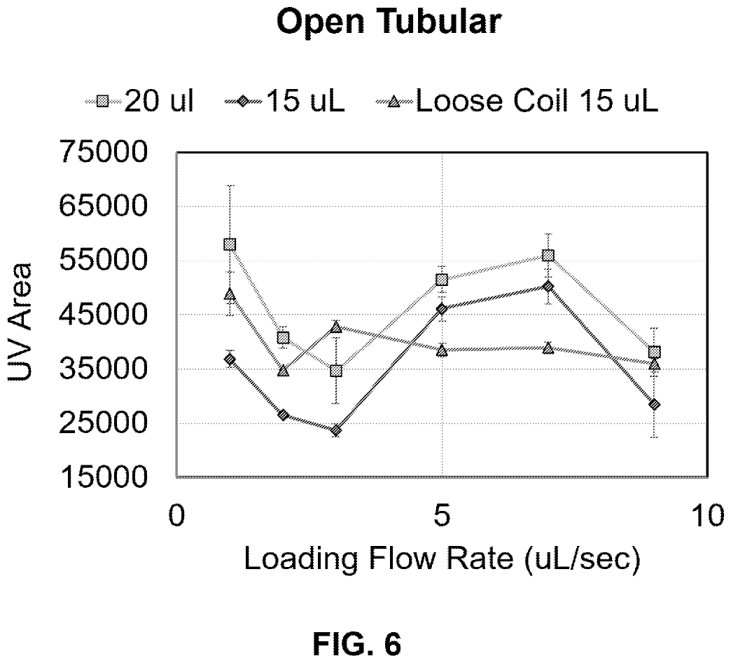

The capacity for the tubular protein capture devices disclosed herein to bind and elute a protein sample was determined for a range of loading flow rates. A tight coil open tubular capture device was connected to a liquid chromatography system with a UV detector. Either 15 uL or 20 uL of sample (IgG) was loaded onto the coil at 1, 2, 3, 4, 5, 6, 7, 8, or 9 uL/sec (loading flow rate). As a comparator, a loose coil capture device was used. As shown in FIG. 6, the tight coil open tubular capture device (square and diamond) provides demonstrable evidence of the effect of Dean vortices, wherein binding is reduced at low flow rates (1-3 uL/sec) below the critical linear velocity needed for the formation of the secondary flows. After a critical point, binding is increased due to the appearance of secondary flows (4-7 uL/sec). Due to back pressure, the system began leaking at loading rates about 8 uL/sec, resulting in a decrease in binding capacity. In contrast, the loose coil (triangle) does not permit secondary flows and exhibits no increase in binding capacity or recovery with increasing flow rate.

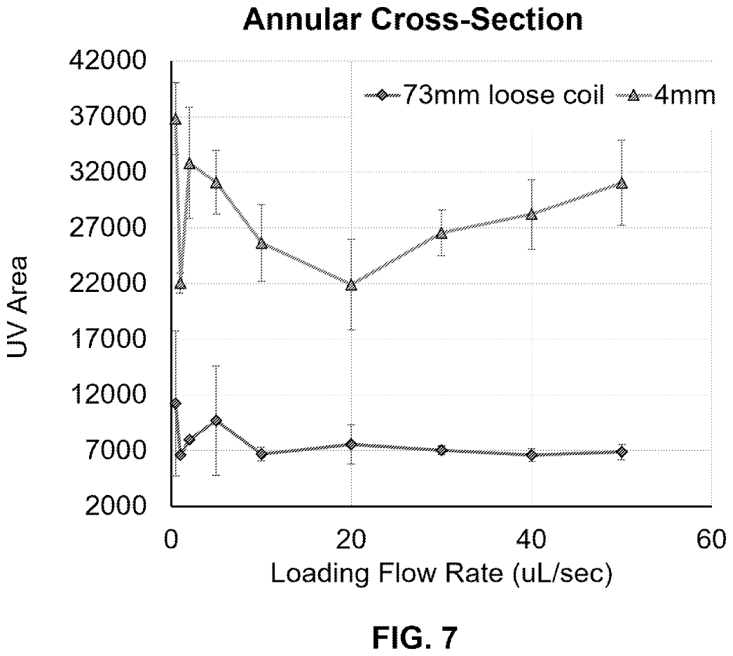

A similar experiment was performed for an annular cross-section capture device. A tight coil annular capture device (4 mm inner coil diameter) was connected to a liquid chromatography system with a UV detector. 20 uL of sample was loaded onto the coil at flow rates between 1-50 uL/sec (loading flow rate). As a comparator, a 73 mm loose coil capture device was used. As shown in FIG. 7, the tight coil annular capture device (triangle) results in a reduction in recovery up to 20 uL/sec loading flow rate, which is consistent with diffusion-limited binding. Above 20 uL/sec loading flow rate, recovery increases, which is consistent with the secondary current improving binding efficiency. The annular capture device is amenable to higher flow rates at equivalent back pressures relative to the open tubular device (approx. less than 150 psi). The 73 mm loose coil capture device (diamond) resulted in significantly lower recovery amounts, with no increase at flow rates above 20 uL/sec.

1. A tubular protein capture device, comprising
(i) a continuous tubular body having an inner surface and an outer surface, wherein the inner surface defines a fluid flow path and includes a protein attached to at least a portion of the inner surface, the protein selected from protein A, protein G, protein L, or combination thereof; and
(ii) an inlet and an outlet for flowing a sample matrix, the inlet and outlet being in fluid communication with the fluid flow path, wherein the continuous tubular body has a helical shape that extends continuously from the inlet to the outlet of the continuous tubular body.

2. The tubular protein capture device of claim 1, wherein the continuous tubular body has an annular geometry.

3. The tubular protein capture device of claim 1, wherein the continuous tubular body has an open tubular geometry.

4. The tubular protein capture device of claim 1, wherein the sample matrix comprises at least one type of protein.

5. The tubular protein capture device of claim 1, wherein the sample matrix comprises a cell culture, a cellular material, a cell extract or combination thereof.

6. The tubular protein capture device of claim 1, wherein the continuous tubular body comprises a plurality of tubes aligned within a hollow enclosure, wherein the inlet and the outlet for flowing a sample matrix being in fluid communication with the plurality of tubes.

7. The tubular protein capture device of claim 1, wherein at least a portion of the inner surface of the tubular body is coated with a polymer and the protein selected from protein A, protein G, protein L or combination thereof is attached to the polymer.

8. The tubular protein capture device of claim 7, wherein the polymer comprises polyethylene glycol, fluorinated ethylene propylene or ethylene tetrafluoroethylene.

9. The tubular protein capture device of claim 7, wherein the protein is attached to the polymer through a covalent bond.

10. The tubular protein capture device of claim 7, wherein the protein is attached to the polymer through a non-covalent interaction.

11. The tubular protein capture device of claim 4, wherein the protein selected from protein A, protein G, protein L or combination thereof is capable of binding to at least one type of protein present within the sample matrix.

12. The tubular protein capture device of claim 1, wherein the continuous tubular body is configured to sustain laminar flow at an apparent Reynolds Number between 3 and 60.

13. The tubular protein capture device of claim 1, wherein the continuous tubular body comprises more than one turns, wherein the distance between consecutive coils is less than 10 mm.

14. The tubular protein capture device of claim 1, wherein the outer surface of continuous tubular body is made of a material selected from metal, glass, polymer or combination thereof.

15. The tubular protein capture device of claim 14, wherein the polymer is selected from a material comprising polyethylene glycol, fluorinated ethylene propylene or ethylene tetrafluoroethylene.

16. The method of capturing at least one type of protein from a sample matrix wherein the sample matrix comprises a cell culture, a cellular material, a cell extract or combination thereof, comprising:
a) providing the tubular device of claim 1;
b) flowing the sample matrix through the continuous tubular body, wherein the protein selected from protein A, protein G, protein L or combination thereof is capable of binding to at least one type of protein within the sample matrix;
c) binding the at least one type of protein within the sample matrix to the protein selected from protein A, protein G, protein L or combination thereof, thereby capturing at least one type of protein from the sample matrix.

17. The method of extracting at least one type of protein from a sample matrix comprising a cell culture, a cellular material, a cell extract or combination thereof, comprising:
(i) performing the method of claim 16; and
(ii) eluting the tubular device with a mobile phase having a pH less than 6.

18. The method of claim 16, wherein the sample matrix comprises less than 100 μg of a total protein.

19. The method of claim 16, wherein the sample matrix flows through the continuous tubular body at an apparent Reynolds Number between 3 and 60.

20. A tubular protein capture device, comprising
(i) a continuous annular tubular body having an inner surface and an outer surface, wherein the inner surface defines a fluid flow path and includes a protein attached to at least a portion of the inner surface, the protein selected from protein A, protein G, protein L, or combination thereof; and
(ii) an inlet and an outlet for flowing a sample matrix, the inlet and outlet being in fluid communication with the fluid flow path, wherein the continuous tubular body has a helical shape that extends continuously from the inlet to the outlet of the continuous annular tubular body.

21. (canceled)
